(19) United States
(12) Patent Application Publication
(10) Pub. No.: US 2008/0038376 A1
(43) Pub. Date: Feb. 14, 2008

(54) ANTI-CANCER COMPOSITION AND METHOD FOR USING THE SAME

(75) Inventor: Brian C. Giles, Scottsdale, AZ (US)

Correspondence Address: SNELL & WILMER L.L.P. (Main), 400 EAST VAN BUREN, ONE ARIZONA CENTER, PHOENIX, AZ, 85004-2202, US

(73) Assignee: PHARMAIONX, INC., Scottsdale, AZ (US)

(21) Appl. No.: 11/753,547

(22) Filed: May 24, 2007

Related U.S. Application Data
(63) Continuation-in-part of application No. 10/469,568, filed on Aug. 28, 2003, now abandoned.
(63) Continuation-in-part of application No. 10/867,115, filed on Jun. 14, 2004, now abandoned.
(60) Provisional application No. 60/808,081, filed on May 24, 2006.
(60) Provisional application No. 60/809,984, filed on Jun. 1, 2006.
(60) Provisional application No. 60/879,857, filed on Jan. 11, 2007.

(51) Int. Cl. A61K 33/00; A61K 33/14; A61K 33/42; A61P 35/00; A61P 41/00

(52) U.S. Cl. 424/722; 424/601; 424/663

(57) ABSTRACT

This invention encompasses an expeditious method and compositions that have been found to show selective cytotoxicity against several different cancer cell lines. for treating a wide variety cancer neoplasms that have tumor microenvironments by administering a stand-alone anti-tumor chemotherapeutic composition or administered as an adjunct with chemotherapy and/or radiotherapy with enhanced tumor site affinity that preferentially elevates the pH at the tumor site to suppress and eliminates the acidic tumor microenvironment, administered as chemosensitizing and/or radiosensitizers enhancing the tumor, suppression, remission and inhibit tumor metastisis. The invention encompasses controlling mechanisms of intracellular and extracellular ionic physiology through the administration of alkali salts for pH modulating and for restoring and enhancing ionic physiology. The compositions are useful in effective treatment of cancers, particularly malignant melanomas and squamous cell carcinomas (SCCs), thereby inhibiting angiogenesis, reducing metastatic proliferation, supressing tumor generated induced acidotic pain.

### CROSS-REFERENCE TO RELATED APPLICATIONS

This application is a continuation-in-part patent application of Ser. No. 10/469,568 filed Feb. 28, 2001 and Ser. No. 10/867,115 filed Jun. 14, 2004 and also claims priority to U.S. Provisional Patent Application having Ser. No. 60/808,081 filed May 24, 2006, U.S. Provisional Patent Application having Ser. No. 60/809,984 filed Jun. 1, 2006 and U.S. Provisional Patent Application having Ser. No. 60/809,984 filed Jan. 11, 2007, all of which are herein incorporated in their entirety.

### FIELD OF INVENTION

This invention is in the field of pharmacology, and relates to anti-neoplastic drugs that include cesium and or rubidium salts for treating cancerous tumors, which are useful as stand alone anti-cancer therapies or as adjunct for radiosensitizing and/or chemosensitizing agents and cancer therapies and procedures in general. More particularly, the present invention is directed to an anti-cancer composition for topical administration which includes a source of cesium ions, a source of rubidium ions, or both.

### BACKGROUND OF THE INVENTION

Drug resistance, either intrinsic or acquired, can result in the ineffectiveness of anti-neoplastic drugs. An example of a mechanism of drug resistance is the expression of the MDR1 gene, which encodes a glycoprotein which acts as an energy dependent multidrug efflux pump in the plasma membrane.

Cancer treatments often involve the use of therapies or methods that are not cytotoxic in themselves, but modify the host or tumor so as to enhance the efficacy of the anticancer therapy. Chemosensitizer efficacy depends primarily upon its ability to enhance the cytotoxicity of a chemotherapeutic drug and also on its sufficiently low toxicity in vivo. Chemosensitization research has centered on agents that reverse or modulate the multi-drug resistance in solid tumors (MDR1, P-glycoprotein). Chemosensitizers known to modulate P-glycoprotein function include: calcium channel blockers (verapamil), calmodulin inhibitors (trifluoperazine), indole alkaloids (reserpine), quinolines (quinine), lysosomotropic agents (chloroquine), steroids, (progesterone), triparanol analogs (tamoxifen), detergents (cremophor EL), and cyclic peptide antibiotics (cyclosporines).

Unfortunately, chemosensitizers benefit only a portion of the patient population. The failure of current chemosensitizers and radio-sensitizers to reverse clinical multidrug resistance may be due to a number of factors which include the following: i) dose levels of the chemosensitizing agent may be inadequate at the tumor site, ii) levels of P-glycoprotein may increase as the tumor progresses, iii) the MDR1 gene may mutate, resulting in decreased binding of the chemosensitizing agent to P-glycoprotein, iv) alternative non-P-glycoprotein mechanisms of resistance may emerge during treatment that are unaffected by chemosensitizing agents and v) a general lack of tumor site affinity (tumor selectivity) for chemosensitizers and their sensitization of normal healthy tissues to the toxic effects of chemotherapy.

In addition, the use of chemosensitizers may have drawbacks. A review of studies where chemosensitizing agents were administered concluded that: i) cardiovascular side effects associated with continuous, high-dose intravenous verpamil therapy are significant and dose-limiting, ii) dose-limiting toxicities of certain chemosensitizers, such as trifluoperazine and tamoxifen, is attributable to the inherent toxicity of the chemosensitizer and not due to enhanced chemotherapy toxicity, and iii) using high doses of cyclosporin A as a chemosensitizer produces hyperbilirubinemia as a side effect.

Accordingly, more efficacious and less toxic chemosensitizers and radio-sensitizers are desperately needed in the physician's art to improve the treatment outcome of chemotherapy patients. The present invention addresses these limitations and provides herein a potential new class of chemosensitizers and chemotherapies and radiosensitizers that permit new approaches and outcomes in cancer treatment.

There is a strong association between skin exposure to the ultraviolet light spectrum from sunlight and development of skin cancers, such as malignant melanoma and the non-melanoma skin cancers, particularly basal cell carcinomas (BCCs) and squamous cell carcinomas (SCCs). The incidence of these varieties of cancers has been rapidly increasing world wide. For example, in Britain there were 4000 newly-diagnosed cases of malignant melanoma in 1994, an 80% increase over the past 10 years. In the United States, approximately 34,100 new cases were anticipated, an increase of 4% per year. Queensland, Australia, has the highest per capita incidence of melanoma globally. However, early detection and widespread public health campaigns and the promotion of the use of sunscreens and reduction of ultraviolet exposure have helped to reduce the number of deaths. BCCs currently affect one in 1,000 in the U.K. population, and the incidence has more than doubled in the last 20 years. One million new cases of BCCs and SCCs are expected to be diagnosed in the USA in 1997, compared to 600,000 in 1990 and 400,000 in 1980.

The most prevalent forms of skin cancers or lesions are the basal cell variety. This variety of cancerous cells that are activated by toxins, radiations, etc., continue growing throughout the patient's lifetime, particularly on the exposed surfaces of the nose and ears, primarily due to being exposed to sunlight on a regular basis. Consequently, the administrating physician will see the therapeutic changes much quicker in the nose and ears than other skin areas.

Over 90% of all skin cancers occur on the noses and ears of individuals that have regularly exposed their noses and ears to sunlight or other ultraviolet radiation. UVB ultraviolet radiation bands are responsible for burning the skin and are also associated with the propagation of malignant melanomas (depletion of the ozone layer of the stratosphere is generally considered to contribute to long-term increases in skin cancer) while UVA ultraviolet radiation bands are associated with premature skin aging and the development of BCCs and SCCs. Childhood sun exposure has been linked to the development of malignant melanoma in younger adults. Other risk factors include a genetic predisposition (fair complexion, many skin moles), chemical pollution, over-exposure to X-rays, and exposure to some drugs and pesticides.

Surgical removal is by far the most common treatment for malignant melanomas, BCCs and SCCs. This can take the form of electrodesiccation and curettage, cryosurgery, simple wide excision, micrographic surgery or laser therapy. Other treatments, used when the cancers are detected at an advanced stage of development, are external radiation therapy, chemotherapy, or to a lesser extent bio-immunotherapy or photodynamic therapy. The choice of therapies is dependent on the type and stage advancement of the disease and the age and health of the patient. All of the prior art therapies suffer from severe limitations.

Prior Art Treatment Modalities

1. Surgical cancer therapy relies on surgical intervention for the removal of the tumor load. There are high risks and stress associated with surgery and post operative complications, high costs, and high risk of life-threatening metastases. It is extremely difficult to be certain that the entire cancer has been completely removed as residual cancer cells frequently survive and metastasize. In one publication, the reported rates for incompletely-excised BCCs was about 30-67%. The immune suppression associated with surgery may also cause any remaining cancerous cells to proliferate, and increases the risk of metastases. In addition, surgery produces pain (acidosis), which requires a separate drug for its management that may contribute to chemical and psychological addiction.

2. Chemotherapy relies primarily on differential toxicity and often does not reach the targeted tumor sites at the tumor edge. It also carries a high risk of immune suppression.

3. Radiation therapy's mode of action causes damage to rapidly growing cells. Unfortunately, radiation also causes permanent genetic damage to the non-cancerous normal healthy viable cells and further contributes to the reduction of pH. Radiation therapy (or radiotherapy) uses ionizing radiation to control malignant cells. It is occasionally used as a palliative treatment for symptomatic relief. Total Body Irradiation (TBI) is prescribed for some types of cancer such as Leukemia. Image Guided Radiation Therapy (IGRT) machines have a CT scanner integrated with the treatment system, or an X-Ray Tube and a Si-detector mounted on a linear accelerator. Immediately before treatment, the patient is scanned and the tumor is located in 3D space. This procedure allows for smaller margins to be used thereby sparing more healthy tissue and escalating the tumor dose. However, radiation therapy can still carry a high risk of immune suppression and poor tumor specificity.

4. Stem cell therapy involves the use of both autologous and (matched) heterologous bone marrow-derived cells for replacing the immune cell population in various types of leukemia and lymphoma. This procedure requires extreme safety measures and is highly stressful for patients. In addition, it is costly and is limited to only a restricted number of malignant diseases.

5. Immunotherapy employs several forms of immune cells isolated from patient's blood (e.g. dendritic cells, lymphokine activated killer cells) which, after in vitro stimulation with tumor antigens or immune modulators, are re introduced to the patient. The intention of this therapy is to enhance the anti-tumor capacity of the immune system. Another approach is to use in vitro incubation of cancer cells from a patient's tumor with natural or artificial immuno modulators (e.g. interferon gamma) in order to induce the expression of membrane molecules (histocompatibility antigens) that can activate those cells from the patient's immune system that are responsible for tumor surveillance and anti-tumor activity. Membrane fragments of these activated cancer cells are re introduced into the patient with the aim to enhance anti-tumor efficacy of the individual's immune system. Limitations of the immunotherapeutic approach include the limited number of tumor types that have successfully been treated (e.g. melanoma, kidney tumors), the expense and complexity of the procedure, and the limited success rates.

6. Gene (Modulation) Therapy is still in its infancy. There are several options, ranging from insertion of specific gene sequences coding for proteins interfering with tumor cell survival (e.g. apoptosis induction, cell cycle arrest) to induction of increased expression of genes involved in the inhibition of angiogenesis by means of natural or artificial modulators of gene activation (no gene insertion). The major limitation to this approach is the difficulty in achieving the required systemic specificity and efficacy.

Most prior art cancer therapies seek to destroy tumors through surgery, radiation or chemotherapy. These therapies are largely based on the following two paradigms: (1) cancer is ‘alien’ tissue and therefore must be removed or destroyed and (2) cancer cells proliferate faster than normal cells and therefore methods and compositions that act on rapidly proliferating cells should destroy cancer. The knowledge gained over the last 50 years of anti-cancer therapies based on these paradigms overwhelming demonstrate that these may be too simplistic for this complex disease. Current anti-cancer therapies, in fact, almost always inflict further injury. If cancer is the result of transformation injury, further injury by the hostile physiological environment within the solid tumors or the hostile electro-physiological environment induced in supporting stromal tissue by practicing the current therapies may serve to exacerbate the original malignant phenotype. This may occur through genomic instability, or due to up-regulation of inflammatory responses, until it progresses in its invasive metastatic form. The multi-parametric approaches are feasible with current magnetic resonance imaging and spectroscopy techniques which provide a high level of versatility in investigating and detecting cancer. With these approaches we can understand the dynamics between the electro-physiological environment, tumor metabolism, tumor invasion, vascularization and metastasis. Such an understanding may provide new paradigms for cancer therapies and increase successful treatment outcomes.

When the tumor has advanced past the stage amenable to surgery, the most common treatment for melanoma or metastatic skin cancers is chemotherapy, which has been largely unsuccessful. The pH within tumor cells (pHi) is similar to (or even more alkaline than) the pH of normal viable tissue cells. The acidic pHe promotes persistent antigenic and metastic signaling, metastatic spread of cancer and neovascularization (including angiogenesis, enhancing blood flow to the tumor mass). The acidic pHe also decreases the efficacy of the immune response to cancer cells. Generally, cancers exhibiting the lowest pHe values are more acidic and more aggressive and hostile (invasive) to the surrounding normal healthy cells and more likely to be fatal to the patient. According to the American Cancer Society, metastasis is responsible for 90% of cancer deaths. Additionally, an acidic hypoxic micro-environment causes genomic instability, and increased resistance to conventional cancer treatment procedures such as multiple drugs, radiation, and chemotherapy.

There are other important consequences of aberrant energy metabolism in cancer cells. As compared to healthy cells, cancer cells have a lower energy charge (ATP/(ADP+Pi)). Additionally, most varieties of cancer cells typically have cellular distributions of ions that are different from normal healthy cells. Neoplastic cancer cells usually contain excess internal sodium and grossly excess internal calcium, often with a deficiency in internal potassium.

The key to using aberrant energy metabolism as a way to enhance site affinity that specifically targets the cancerous tumor cells' microenvironment that produces measurable tumor selective alkalinization involves administering an alkaline composition that has little toxicity to normal healthy viable cells which preferentially targets and elevates the pH level of the tumor micro-environment, thus causing the non-viability and apoptosis of tumor cells. This leads to the elimination of tumors and metastases and the electro-physical environment forces the cancer cells to die. The composition of the present invention reduces and eliminates the hypoxic acidification produced by the cancer cells so that the physiologic pHe range is modulated (elevated) and approaches a physiological optimum range between 7.31 to 7.45. This enables the anti-cancer activities of the immune system to function in a more optimal osmotic biochemical, ionic and electro-physiological environment.

The prior art methods and compositions for treating cancer often adversely affect ionic function and interfere with critical pH ranges. Some of these therapies often contribute to a further reduction of the systemic pH thereby further promoting acidic tumor micro-environment and compromising the patients' survival and recovery rate. A far better strategy is to provide therapies that alter the tumor cells ability to transport H+ and other ionic species across its membrane. This approach provides site affinity and selective modulation (elevation) of the tumor site and of the tumor pHe (microenvironment) enabling elevation of the pHi of cancer cells that is outside the viability zone of the cancer cells.

This elevation of the tumor microenvironment promotes the formation of an electro-physical barrier to resist pH reduction and provides resistance to tumor formation and proliferation and invasion, metastasis seeding, and localized neovascularization. Such a therapy that takes into account the consequences of low pHe and pHi and that increases tumor pHe and tumor pHi without causing serious side effects provides significant improvements over the prior art. The composition and method of the current invention is separate from and superior to the above-referenced prior arts by eliminating the short and long term toxic side effects so prevalent in the prior art methods and compositions.

### SUMMARY OF THE INVENTION

An object and advantage of the present invention is to provide a therapeutic composition with tumor site affinity that selectively modulates (elevates) the tumor's micro-environmental (pHe), which typically ranges from 6.70 to 6.80, to a more physiologically optimum pHe range from about 7.31 to 7.45 or slightly higher to bring the tumor cells out of their viability zone such that they die in a predictable and controllable rate.

Another object of the present invention is to provide an anti-cancer composition and method that can be cost-effectively administered as a stand-alone topical chemotherapy or employed as an adjunct in conjunction with a wide variety of conventional cancer therapies.

A further object of the present invention is to provide a composition that can be employed as a topical chemosensitizer in reversing drug resistance by preferentially targeting and elevating the tumor micro-environment to enhance the electro-physical environment for therapeutic gain for a wide variety of tumor types. The composition of the present invention acting as a chemosensitizer increases the effective values of chemotherapeutic drugs and can be used in both in vivo and in vitro applications.

Still another object of the present invention is to provide a topical treatment which can help prevent or treat metastatic tumors at the primary tumor site with enhanced site affinity for malignant cells that is not restricted by the type of cancer cell.

Yet another object of the present invention is to provide a rapid testing process and procedure ranging from about 24 to 48 hours to clinically verify the efficacy of the inventive composition in a patient suspected of or diagnosed with cancer and to verify the efficacy of the composition in an individual patient's particular cancer or cancers to determine the doses, times, and the regimens for favorable outcomes.

The cancer therapy and composition for using the same of the present invention involves administering a therapeutically effective and non-lethal amount of a pharmaceutical composition to mammals in need of such a therapy and more specifically humans suffering from cancerous neoplasms and to prevent the formation and elimination of the hostile cancer viability zone and more specifically the tumor micro-environment which can help treat metastatic tumors and at sites other than a primary tumor site with site specificity for malignant cells that is not restricted by the type of cancer cell, including damaged or necrotized cells and tissues. “Inherent bio-localization” for targeted in vivo and vitro delivery means having specificity for targeted sites for damaged tissues.

The method and composition may be administered as a therapy to obtain complete remission. The method employs administering a virtually nontoxic composition to electrophysically suppress and eliminate the formation of the electrophysiological viability zone of cancers.

The method and composition described in the invention have several related effects on the development and propagation of cancer's micro-environment. The current inventive composition interferes with the hypoxic acidic dependent energy metabolism of the cancer cells. This effect renders the cancerous cells less able to supply the energy required for their rapid proliferation that is typical of cancerous tumors thereby resulting in the reduction or elimination in the viability zone of the cancer cells. Secondarily, the composition reduces localized acidification (preferentially in the tumor micro-environment) and increases oxygenation, eliminating the adverse effects caused by acidic hypoxia.

One exemplary embodiment of the present invention is directed to an anti-cancer composition for topical administration which includes a cesium ion source and/or a rubidium ion source and a carrier suitable for topical application. For topical administration, the key advantage is delivering the composition in a lowered or suitable viscosity range so that it serves as a penetrating agent. For example, a composition of the present invention having a viscosity at or below about 36 dynes per cm² will seep in between the skin cells and may also be transferred osmotically from cell to cell and is capable of entering the subcutaneous tissues. The viscosity range between the inside of the cell to the outside of the cell is about 36 dynes per cm² depending upon the cell hydration and other factors.

In one aspect of the exemplary embodiment of the inventive composition, the composition may include a pH within a range of about 6.7 to 7.2. In another aspect of the exemplary embodiment of the present invention, the carrier may include a liquid, a gel, a cream, an ointment, a lotion, a paste, en emulsifier, a solvent, a liquid diluent, a powder, or any other medium suitable for topical application. In yet another aspect of the exemplary embodiment of the inventive composition, the composition may include an ORP within a range of about −1 m.v. to about −50 m.v. The inventive composition may also further include a surfactant or surfactants.

Skin Type For Topical Administration For Variable Skin Types

Another exemplary embodiment of the current invention encompasses a method and composition to be topically administered or applied to a patient having cancer with skin that is thin (or very thin) that is adjusted with a suitable surfactant for the corresponding penetration such that the composition has a viscosity ranging between about 15 to 25 dynes per cm² and a pH range of about 6.70 to 7.20.

Yet another exemplary embodiment of the current invention encompasses a method and composition applied to a patient having cancer that has normal skin thickness that is correspondingly adjusted to penetrate with a suitable surfactant where the composition has a viscosity ranging between about 15 to 20 dynes per cm² and a corresponding pH range of about 6.70 to 7.20.

Still another exemplary embodiment of the current invention encompasses a method and composition applied to a patient having cancer that has thick skin and/or very deep tumors that is adjusted with a surfactant such that the composition has a viscosity ranging between about 8 to 20 dynes per cm² and a pH range of about 6.70 to 7.20, more preferably ranging between about 10 to 15 dynes/cm².

In yet another exemplary embodiment of the invention, the composition may include cesium citrate as the cesium ion source with a concentration of 1/10 of one percent and the composition may be sufficiently adjusted to a near neutral pH with a viscosity of about 33 dynes/per cm or lower, ranging from 8 to 30 dynes per cm².

Cesium and or rubidium ions alter the ionic physiology of the cancer cell, including inhibition of trans-membrane movement of potassium. Cesium and rubidium are effective for the control of potassium fluxes and linked hydrogen ion (H+) and other fluxes that act on all acidic-dependent cancers and provide site affinity or site directing to selectively elevate the pH of the tumor micro-environment. This provides a tumor selective modulation which elevates the tumor pHe and pHi to the physiological optimum range that is outside of the tumorous cancer's viability zone.

The composition cesium and/or rubidium ions provide site directing ions for targeted in vivo delivery. “Bio-location”, or “Site-directing”, “site-directed” or “site affinity” means having specificity for targeted sites. “Specificity for targeted sites” means that upon contacting the cesium and rubidium ions with the targeted site, for example, under physiological conditions of ionic strength, homeodynamic balance, temperature, pH and the like, specific binding will occur. The interaction may occur due to specific electrostatic, hydrophobic, entropic or other interaction of certain residues of the conjugate with the specific targeted tumor microenvironment residues to form a stable complex under conditions effective to promote the interaction.

In another exemplary embodiment of the present invention, the cesium ion source in the anti-cancer composition for topical administration includes a cesium salt selected from at least one of the following: The cesium salts included in the composition of the present invention may be formed using a variety of acids, including, but not limited to: Carbonate, Chloride, Citrate, Malic, Malate, Nitrate, Phosphate, Sulfite, and Sulfate. The carbonate, citrate and sulfate salts are safer than other salts and sulfate and citrate are the preferred forms. Phosphate is relatively safe but may interact with calcium and the chloride form should be used with caution. In yet another exemplary embodiment of the present invention, the cesium salt may include a combination of cesium sulfate and cesium citrate in a ratio of about 3:2. An even more optimum ratio includes between about 2 to 3 cesium sulfate to about 1 cesium citrate. In still another exemplary embodiment of the present invention, the cesium ion source and/or rubidium ion source in the composition may comprise a range of about 1,000 ppm to 150,000 ppm. The total combination of cesium salts preferably comprises from about 0.5% to 5% of the composition of the present invention.

The inventive composition provides an enhanced electrophysical barrier to tumor growth, tumor invasion, metastasis seeding, and localized neovascularization for therapeutic gain for all stages of malignancies including some cancers that are resistant to conventional therapies. The composition bypasses the cancer cell's mechanisms of resistance against conventional anti-cancer drugs at dosages which are less toxic to normal healthy viable cells than the currently available anti-cancer drugs.

In another exemplary embodiment of the invention, the anti-cancer composition for topical administration may include a soothing agent such as a menthol, mentholyptus, or a eucalyptus and/or an electrolyte which includes one or more of sodium, potassium, calcium, chlorate, magnesium, bicarbonate, phosphate, and sulfate. The composition may also include at least one or more of Vitamin B6, Vitamin B12, Vitamin D2, Vitamin D3, manganese, an antibacterial, and antifungal agent, and an anti mold agent.

The exemplary anti-cancer compositions of the present invention may also be used as adjuncts with one or more of the following: a surgical procedure, a laser therapy, a micro-radiation pellet implant, a cryogenic procedure, a chemotherapy, a radiation therapy, a electrodessication/curettage, a laetrile therapy, an immuno-therapy, and an enzyme therapy.

The present invention is also directed to a method for preventing or treating cancer which includes topically administering an anti-cancer composition for topical administration which includes a cesium ion source and/or a rubidium ion source, and a pharmaceutically acceptable carrier suitable for topical application. The method for treating cancer may also include the step of administering a cesium chelator and/or a rubidium chelator. The step of administering a cesium chelator and/or a rubidium chelator may include the step of administering one or more of omega 3 oil, cod liver oil, Vitamin E, and Vitamin A.

The present invention also includes a method for treating a wound, a sore, a pain, an infection, an inflammation, a scar, or a dermatological condition which includes the topical administration of an anti-cancer composition which includes a cesium ion source and/or a rubidium ion source, and a pharmaceutically acceptable carrier suitable for topical application.

The present invention is also directed to a composition for enhancing the toxicity of a topically administered chemotherapeutic agent which includes a cesium ion source and/or a rubidium ion source, and a pharmaceutically acceptable carrier suitable for topical application.

### DETAILED DESCRIPTION

### Definitions

As used herein the terms “a therapeutic agent”, “therapeutic regimen”, “radioprotectant”, and “chemotherapeutic” mean conventional drugs and drug therapies, including vaccines, for treating cancer, viral infections, and other malignancies, which are known to those skilled in the art. “Radiotherapeutic” agents are well known in the art.

As used herein, “complete remission” or “remission” is defined as having normal blood pictures accompanied by no clinical manifestations of cancer and other immunological markers that are indicative of cancer.

As used herein viscosity is measured in dynes per cm2 at 20° Centigrade.

As used herein acidity and alkalinity are measured by pH which is defined as the negative logarithm of the hydrogen ion activity: pH=−log(H). The parameter pHe is the pH on the exterior of the cell and pHi is the pH on the interior of the cell.

As used herein the term “tumor micro-environment” refers to both the non-cellular area within the tumor and the area directly outside the tumorous tissue but does not pertain to the intracellular compartment of the cancer cell itself.

As used herein, “about” is defined as plus or minus 2.5%, unless specifically stated otherwise.

As used herein, the term “a method for treating cancer” means that the disease and the symptoms associated with the cancer are alleviated, reduced, ameliorated, prevented, placed in a state of remission, or maintained in a state of remission.

As used herein, “pharmaceutically acceptable carrier” includes any and all solvents, surfactant(s), dispersion media, coatings, antibacterial and antifungal agents, isotonic and absorption delaying agents, and the like. The use of such media and agents for pharmaceutically active substances is well known in the art. Except insofar as any conventional media or agent is incompatible with the active ingredient, its use in the therapeutic compositions is contemplated.

As used herein, “cesium sulfate” also means dicesium sulfate, sulfuric acid, and discesium salt.

As used herein, “cesium citrate” also means 2-hydroxy-1,2,3-propanetricarboxylic acid salt.

As used herein, “glycerol” also means glycerin, glycerine, propane-1,2,3-triol, 1,2,3-propanetriol, 1,2,3-trihydroxypropoane, glyceritol, and glycol alcohol. It is a sugar alcohol that has three hydrophilic alcoholic hydroxyl groups (OH) that are responsible for its solubility in water. Glycerol has a prochiral spatial arrangement if atoms and has a wide range of applications.

As used herein, “DMSO” also means dimethyl sulfoxide, methyl sulfoxide, and methylsulfinylmethane. The molecular formula for DMSO is C₂H₆OS. Related compounds may also be used which include diethyl sulfoxide, dimethyl sulfoxide, dimethyl sulfone, and acetone.

For the purposes of this specification it will be clearly understood that the word “comprising” means “including but not limited to”, and that the word “comprises” has a corresponding meaning.

Normal healthy human cells optimally function in a pHe range between 7.31 to 7.45 and have a typical pHi ranging between 6.60 to 6.80. The common denominator for almost all solid cancerous tumors (neoplasms) is that they require a reduced extracellular pHe (tumor microenvironment) which is more acidic than the intracellular pHi. Virtually all neoplasms have a narrow viability zone with a tumor-microenvironment having a pHe ranging from about 5.50 to 7.21, and generally ranging from about 6.60 to 6.80 leading to an acid-outside plasma level pH gradient in the tumors. This acidotic outside pH gradient in tumors can exert a protective effect upon the cancerous cells particularly from weak acid-based anti-cancer drugs.

The present invention is generally directed to a method for treating cancer and compositions for topical administration which include a cesium ion source and/or a rubidium ion source and a carrier suitable for topical application. The anti-cancer method and compositions may be used therapeutically, as well as diagnostically, when coupled with, e.g., radioactive agents, proton capture agents, or other tumor toxic physical or chemical agents. These toxic substances are preferentially localized in the tumor microenvironment (as compared to their adjacent normal cells) through the specificity of attachment of the compounds. This site affinity or site directing (tumor selectivity) is universally recognized as a crucial factor for achieving effective anti-tumor therapy, and a factor which has currently not successfully been achieved. Cesium and/or rubidium ions have efficacy in site affinity which enhance their use as new therapeutic drugs and/or as adjuncts for other cancer therapies.

The composition of the present invention is particularly useful in site directing (preferentially elevating the pH of the tumor micro-environment) of a wide variety of solid tumors that have a pHe in the tumor microenvironment that ranges from about 5.20 to 6.90, selectively elevating the tumor microenvironment outside the cancer's viability zone to about 7.40 to 7.50 and occasionally higher.

The low extracellular pHe is a consequence of tumor growth and is aggressively hostile to the growth and survival of normal viable cells. This low pH promotes tumor invasion and perfusion and further contributes to tumorigenic transformation, particularly where tissue had been subjected to mechanical trauma. This suggests a direct and possibly causative correlation between locally inadequate blood flow and localized acidosis and elevated tissue acidity, which in turn promotes cancer formation and a propagation environment.

Cesium and/or rubidium ions preferentially increase the alkaline ionic concentration in the tumor microenvironment, thus elevating tumor pHe to a more physiologically optimum range for fast tumor suppression and remission. Such a response is instrumental in reducing and eliminating tumor generated acidotic induced pain. The composition of the present invention can alleviate, reduce, ameliorate, or place or maintain in a state of remission the clinical symptoms or diagnostic markers associated with cancer. A mixture of salts containing cesium ions is preferred.

The composition of the present invention promotes ionic changes in the pHe and pH by modulating cancer microenvironmental conditions that simultaneously enhance the ability of normal healthy viable cells and normal tissues surrounding the tumor to tolerate and resist decreased pHe and decreased pHi and to resist the transport of H+ efflux across cell membranes. The composition of the present invention promotes an electrophysical barrier to tumor invasion, eliminates the cancer viability zone (formation and propagation environment) which are a prerequisite for tissue invasion and metastatic seeding, eliminates the aberrant energy metabolism necessary for the viability of cancer formation and survival, and interrupts the transition from non-invasive premalignant to invasive malignant morphology including the acquisition of angiogenesis, increased glucose utilization and increased lactic acid and/or carbonic acid production.

In one embodiment of the present invention, the method and compositions can be used to determine the efficacy of the anti-cancer therapy in a particular patient through testing and observations relating to ionic anti-cancer physiology. Clinical observation and diagnostic testing are used for adjustment of the non-lethal dosages.

A large number of cancer deaths are due to secondary infections, primarily from bacteria invasion. If a patient's immune system is suppressed by acidosis, such as bacterial-induced or tumor-generated acidosis, then the compositions of the present invention described herein will contribute to enhancing the functioning of the immune system and will promote resistance to secondary infections including helping the immune system fight the cancer.

The composition of the present invention suppresses a wide variety of infectious microorganisms, i.e. bacteria, which often have acidotic energy metabolisms. This is advantageous in that antibodies and phagocytes cannot work effectively in tissue having increased acidity, i.e. a reduced pH.

Preferably, the composition(s) of the present invention are able to inhibit the growth of at least one cell line selected from the group consisting of, but not limited to the following: MM96L, MM229, MM220, MM237, MM2058, B16, LIM1215, HeLa, A549, MCF7, MCC16 and Colo16. More preferably, the compound(s) is able to inhibit growth of, or to induce differentiation in, MM96L cells. In a second aspect, the invention provides a composition comprising an active compound as described herein, together with a pharmaceutically-suitable carrier or surfactants(s). The composition and method of the present invention may be applied to a cancer which comprises a solid tumor. The method and composition of the present invention are particularly effective against cancers selected from the group consisting of malignant melanoma, other skin cancers including Merkel cell carcinoma, squamous cell carcinoma and basal cell carcinoma. Additionally, the invention provides a method of inhibiting proliferative activity of neoplastic cells which comprises the step of exposing the cells to an anti-proliferative amount of a compound of the invention. The cells may be treated either in vitro or in vivo.

Method of Manufacture

Principal Active Ingredients

The cesium salts included in the composition of the present invention may be manufactured using a wide variety of acids, and may include, but are not limited to, one or more of the following: The cesium salts included in the composition of the present invention may be formed using a variety of acids, including, but not limited to: Carbonate, Chloride, Citrate, Malic, Malate, Nitrate, Phosphate, Sulfite, and Sulfate. The carbonate, citrate and sulfate salts are safer than the other salts and sulfate and citrate are the preferred forms. Phosphate is relatively safe but may interact with calcium and the chloride form should be used with caution.

The cesium salts which include carbonate, chloride, citrate and sulfate are generally safer. Cesium phosphate is relatively safe but may interact with calcium. Cesium chloride may mask cell differentiation (separating cancer cells from healthy cells). In addition, excessive doses of cesium chloride combined with exposure to light may promote a caustic effect to healthy skin cells.

Cesium citrate is the preferred form of cesium salt for use in the topical composition of the present invention. Cesium citrate alone may have significant anti-cancer application. Cesium sulfate has long term benefits in maintaining remission and produces minimum damage to healthy cells and tissues. Cesium carbonate provides a slightly more neutral range in combination with the cesium sulfate.

A preferred combination of cesium salts for use with the composition of the present invention includes a combination of two cesium salts, namely cesium sulfate and cesium citrate in a ratio of about 3 cesium sulfate to about 2 cesium citrate. An optimum ratio of cesium sulfate to cesium citrate includes a ratio of between about 2 to 3 cesium sulfate to about 1 cesium citrate.

The inventive composition acts on a mucous level and a pH level, and/or a genetic level, and also provides the effective resonance for the optimum vibration level of anti cancer cell activity. Care should be taken in determining dosage because excessive dosage of the topical composition of the present invention can be detrimental in terms of the free radicals that are produced. Excessive doses that build up cesium in the lymph and incorrect cesium salt ratios should be avoided.

Additionally, other cesium and rubidium salts might be used in a wide variety of topical compositions, such as, but not limited to, various organic or metallic salts, if they meet the following requirements: (1) they must be pharmaceutically acceptable and have an acceptably low level of toxicity; and (2) they must have sufficiently high levels of cationic dissociation to allow the remaining negatively charged ions to effectively reduce acidity, including the acidity of tumor cells and skin lesions.

Another exemplary embodiment of the present invention includes a composition having cesium and/or rubidium ions in addition to ingredients that enhance ionic pH physiology. Examples of such ingredients include electrolytes (saline compounds) such as potassium, sodium, and magnesium. Potassium, and other major electrolytes (e.g. sodium, calcium, chloride, bicarbonate, phosphate, and sulfates) are preferably added to the formulation in proportion to the potassium. Other ingredients that may be included to effectively potentiate the tumor site affinity for cesium/rubidium ionic action include manganese, Vitamin B6 (pyrodoxine), Vitamin D2, Vitamin D3, and/or Vitamin B12.

Surfactants

Surfactants can be used as penetrating agents, dispersing agents, solubilizing agents and spreading agents. Suitable surfactants for the present invention which act as penetrative agents are those which are reasonably stable throughout a wide pH range, including non- and amphoteric organic or synthetic detergents. Non-petrochemical surfactants are preferred. Some examples of suitable surfactants include, but are not limited to polysorbates such as Tween 40 and Tween 80 (Hercules); sorbitan stearates, sorbitan mono-oleate, etc.; sarcosinates such as sodium cocoylsarcosinate, sodium lauroyl sarcosinate (Hamposyl-95 ex W. R. Grace); cationic surfactants such as cetyl pyridinium chloride, cetyl trimethyl ammonium bromide, di-isobutyl phenoxy ethoxy ethyldimethyl benzyl ammonium chloride and coconut alkyl trimethyl ammonium nitrate.

Other suitable surfactants (penetrative agents) are disclosed by Gieske et al in U.S. Pat. No. 4,051,234. Examples of these surfactants include, but are not limited to, alkyl sulphates; condensation products, fatty alcohols, fatty amides, polyhydric alcohols (e.g. sorbitan monostearate, sorbitan oleate), PEG (polyethylene glycol) 400; Sodium lauryl sulfate; sorbitan laurate, sorbita palitate, sorbitan stearate available under the tradename Spans® (20-40-60 etc.); polyoxyethylene 20 sorbitan monolaurate, polyoxyethylene (20) sorbitan monopalmitate, polyoxyethylene (20) sorbitan monostearate available under the tradename Tweens® (polysorbates, 20-40-60 etc); and Benzalkonium chloride. The preferred surfactant(s) are glycerine and DMSO. DMSO is added to the glycerine to further adjust (lower) the viscosity.

The purpose of using surfactants in the preferred composition of the present invention is to adjust the surface tension in dynes per cm² (at 20° C.) of the compositions so that they can function as penetrating agents in order to maximize the amount of cesium and/or rubidium ions deposited in or near the nucleus of the cancerous cell i.e. tumor(s).

In a preferred embodiment, the surface tension of the solution is between about 8 to 50 dynes/cm, in order to yield a penetrating agent and/or carrier composition having a preferred surfactant within the range from about 8 to 36 dynes per cm². The use of such a topical composition has minimal systemic side effects. Surface tension of a given formulation may be adjusted by adding a surfactant(s) in addition to the active ingredients in order to bring it into the preferred range(s).

The HLB (hydrophille-lipophile-balance) is used to describe the characteristics of a surfactant. This arbitrary system consists of a scale to which HLB values are determined experimentally and assigned. If HLB value is low, the number of hydrophilic groups on the surfactant is small, which means it is more lipophilic (oil soluble). Surfactants can act as a solubilizing agent by forming micelles. For example, a surfactant with a high HLB would be used to increase the solubility of an oil in a medium. The volume of dilution fluid will vary according to the total dose administered and the required dynes per cm² range.

It is contemplated that formulations according to the present invention will preferably have a pH in the range of about 6.60 to 8.00; an osmotic pressure of the solution between about 100 mOsm/kg to 700 mOsm/kg; and a NaCl equivalency to the solution is preferably between about 0.5% NaCl to 4.0% NaCl.

To achieve effective deposition of medication within the tumor(s) it is preferable to adjust the surface tension of the composition for topical administration with surfactants to between 8 to 50 dynes/cm², more preferably between about 10 to 30 dynes/cm², and most preferably between about 15 to 25 dynes/cm².

Appropriate compositions for this purpose may be formulated by using surfactants, NaCl or other chemicals entities to adjust the solution for administration to have the following properties: i) surface tension preferably between about 8 to 50 dynes/cm², more preferably between about 10 to 30 dynes/cm², and most preferably between about 15 to 25 dynes/cm², ii) osmotic pressure between about 350 mOsm/kg to 800 mOsm/kg, more preferably between about 450 mOsm/kg to 700 mOsm/kg and most preferably between about 450 mOsm/kg to 600 mOsm/kg, and iii) pH preferably between about 6.60 and 8.00, but it may vary according to the properties of the medication used.

The surface tension and, to a lesser degree, the salt ratios are critical factors in getting optimal deposition of the composition into tumors in the skin. Having a surface tension that is too low increases permeation into and past tumor cells and into the circulatory system. This should be avoided. In contrast, if the surface tension is too high, much of the medication is not deposited within the tumors. Glycerin may be both a surfactant and a carrier in the composition of the present invention.

Secondary Ingredients

Secondary ingredients are chosen to complement or potentiate the action of the cesium and or rubidium ions. The examples of enhancing secondary ingredients are given to instruct the physician in the principals of their selection and are not intended to exclude other ingredients not mentioned or as needed by a specific patient.

Enhancement of cesium and/or rubidium therapy can be accomplished by inclusion of ingredients that enhance the shift (elevate) towards apoptosis induced by ionic physiology. Examples are compounds that stimulate calcium accumulation, such as calcium supplements with potassium and magnesium, preferably in a 2 to 1 ratio, preferably with an equal portion of Vitamin D3 combined with Vitamin D2, such as but not limited to calcium, with potassium and magnesium preferably administered somewhat independently or alternatively in a ratio of 2× or 4× as much potassium as magnesium if necessary, and 2× as much as magnesium as calcium, and 4× as much calcium as the potassium, and a small amount of manganese. Administering excessive doses of potassium should be avoided as it may cause arrhythmic responses in the heart of some sensitive patients In addition, administering excessive doses of calcium should also be avoided.

Another class of ingredients which potentiate the activity of the cesium and rubidium ions are those which stimulate or support the immune system and normal healthy viable cells, especially those which may be deficient as a secondary consequence of cancer. These include potassium, magnesium, manganese, Vitamin D2, Vitamin D3, Vitamin B6 (Pyrodoxine) and B12, and other ingredients that complement the salts of cesium and/or rubidium therapy which may be useful in reducing cancer viability or reducing the toxic side effects from cancer therapies. Compounds intended to combat secondary infection such as antibiotics with antiviral, anti-bacterial, antifungal, and anti mold action may also be included if appropriate.

When administering larger or maximum doses of the inventive composition, the composition should be administered sequentially with calcium, potassium, magnesium, or manganese. Alternatively, the inventive composition may be combined with systemic administration therapy or other routes or as a stand alone anti-tumor therapy chemosensitizer or as an adjunct with radiation therapy as a radiosensitizer.

Cesium seeks to find its counter-ions to form salts. In the process it nullifies some of the DNA and RNA disruption factors that take place in a variety of cancers. Cesium forms less optimum structures in the cancers and destabilizes the cancerous tissues i.e. neoplasms and solid tumors.

The frequency with which the cesium ions strike the cancer cells in a segment of its DNA chain that affects the cancerous tissue. Rubidium ions are less site-directed and therefore burn more cells and tissues. Cesium ions are more efficiently site directed to the tumor site than rubidium ions.

Physical Form

Modes of Manufacture

After determining the medications to be used in the topical composition, each ingredient is weighed/measured out individually, added together and dissolved in a sterile glycerin and DMSO composition for administration. The preparation is then tested to ensure that it is within the parameters established for surface tension, osmolarity, pH, and sodium chloride equivalency. This is done by using the appropriate equipment for each test. To prepare a unit dose, the ingredients of such formulations generally will be dissolved in a solvent such as glycerin and DMSO or other suitable solutions.

For the purposes of preparing formulations according to the present invention. the pH of the various solutions may need to be adjusted to achieve stability or increase effectiveness. When necessary, the pH is adjusted by adding buffering agents to arrive at the most preferable range of pH needed for a particular tumor or skin type. The surface tension may also be adjusted using surfactant(s) to fall within a preferred range in dynes/cm². The topical composition of the present invention is light sensitive.

Modes of Administration

The composition may take the form of gels, oils, creams, ointments, bandages, impregnated plastic strips, patches, transdermal patches, dressings, gauze, topical lotions, transdermal patches, etc. The compositions may also be presented in unit-dose or multi-dose containers, for example sealed ampoules and vials.

Inclusion Criteria

Inclusion criteria for the method and composition of the present invention include: 1) patients who show verifiable tumor control or shrinkage within 24 to 48 hours after administration of the initial testing dosage of the inventive composition, 2) patients who are unable to attain complete remission with cytotoxic agents, and 3) patients in complete remission after conventional treatment who show residual cancer cells, indicating likelihood of relapse.

The method and topical composition of the present invention encompasses a testing procedure for testing patients with suspected or diagnosed cancer with the method and composition. It also includes determining the therapeutic gain for tumor control and remission in an individual patient with a particular variety of cancer or cancers and stage of advancement to actually help determine the predictable outcomes of using the current inventive methods and compositions in an individual patient including doses, routes, durations and resting periods.

As an example, testing can be performed to measure the efficacy of the composition (tumor shrinkage) after administration of the composition to the patient between 24 to 48 hours, more preferably testing is performed between 24 to 48 hours after the initial administration of the composition.

Efficacy

Administration of a dosage regime that results in site directed selective alkalinization of the pHe of the tumor micro-environment and a normal, as opposed to cancerous, ionic response to glycolytic metabolism are the short-term indicators of the effectiveness of the inventive compositions. The method and composition is effective for treating large volume solid tumors that must be confirmed by tumor regression. A lack of adequate response by the tumor pHe modulation and other indicators suggests dehydration and/or insufficient dosages.

Treatment Protocols

The method and composition of the present invention can be used alone or in combination to act as a sensitizer for chemotherapeutics, radioprotectants, radiotherapeutics, and radiosensitizers to either improve the quality of life of the patient, or to treat cancer. For example, the inventive topical composition can be used as an adjunct administered before, during or after the administration of one or more known antitumor agents. (See, for example, the Physician Desk References 1997). In addition, the inventive topical composition can be used before, during or after a wide variety of radiation treatments. Sequential administration is preferred.

Topical Therapy and Protocol

The inventive topical composition of the present invention provides a method and composition for preventing or alleviating damage to skin, which includes, but is not limited to, damage caused by ultraviolet irradiation, ionizing radiation, exposure to ozone, microwave radiation, or the like. The method comprises the step of topically administering an effective amount of a compound of the present invention to a subject in need of such treatment. This aspect of the invention may be used in the treatment of solar keratosis, skin damage occurring during radiotherapy, and the like.

The invention provides a method of stimulating proliferation of non-neoplastic cells which comprises the step of exposing the cells to a proliferation-inducing amount of a compound or a composition of the current invention. This is useful in inducing regeneration of cells and tissues and, because T-lymphocytes may proliferate in response to the compositions of the invention, it is useful in promoting the immune response to disease states.

While it is particularly contemplated that the compounds of the invention are suitable for use in medical treatment of humans, it is also applicable to veterinary treatment, including treatment of companion animals such as, but not limited to, dogs and cats, and domestic animals such as horses, cattle and sheep, or zoo animals such as felids, canids, bovids, and ungulates. Dosage is at the discretion of the attendant physician or veterinarian, and will depend on the nature and state of the condition to be treated, the age and general state of health of the subject to be treated, the route of administration, and any previous treatment which may have been administered.

The present invention is effective in ameliorating damage from solar UV and like agents by enhancing DNA repair and the immune response either in the target or effector cells. On the basis of the gene expression array data, the compounds of this invention are expected to have activity in modulating gene expression in the G-protein, PKC and Ras signaling pathways in a manner that leads to anticancer activity in vivo. The present invention also has use as an adjuvant to radiotherapy or to therapy with other DNA-damaging agents on the basis of down-regulation of protective proteins (GADD45 and DAP3).

The administered dosages of the inventive composition depend on the type of the tumor and its location in the dermal layers of the body, the volume and mass of the tumor, and the therapeutic effects on the tumor from the composition. A physician evaluates the effect of the inventive composition with medically appropriate methods to verify the degree of tumor shrinkage and/or pH modulation (elevation) of the tumor microenvironment. There should be measurable tumor control and shrinkage within about 48 hours from the initial administration. In the event of an adverse reaction to the inventive composition, calcium should immediately be administered to the patient. Alternatively, the calcium can be administered sequentially or after about 6 hours after administration of the initial cesium and/or rubidium.

The topical composition of the present invention is preferably in a low or suitable viscosity range. For example, a composition having a viscosity at or below about 36 dynes per cm² will seep in between the cells and is transferred osmotically from cell to cell thereby enabling the composition to penetrate into the subcutaneous tissues. The viscosity transition range between the inside of the cell to the outside of the cell is about 36 dynes per cm² depending upon the cell hydration and other factors. The preferred composition's viscosity range for topical application is below 36 dynes per cm².

For topical administration, the composition preferably consists of a combination of two cesium salts, namely cesium sulfate and cesium citrate in a preferred ratio of about 3 cesium sulfate to 2 cesium citrate. A more optimal ratio may include about 2 to 3 cesium sulfate to about 1 cesium citrate. Cesium sulfate salt promotes less free radical damage to the cell tissues, is more neutral to the normal healthy cells, is detrimental to the cancer cells, and further contributes to the cell differentiation between the two types of cells. Cesium sulfate is also more effective in terms of safety for longer term administration and for maintaining a state of complete remission.

The physician should administer a topical dose of the composition of the present invention sufficient for a balanced reaction on a molecular basis and should avoid excessive doses that permeate past the needed area. For example, a practical calculation would be no more than one cesium and/or rubidium ion per cancer cell. The physician should also avoid administering the composition into a lesion for a prolonged period (i.e. over a period of 1 to 2 consecutive days). After the predetermined time period for administration, the physician should remove any of the topical composition from the affected area.

An exemplary embodiment of the invention encompasses the treatment schedule and timing of the administration of the topical composition, including an administration period, a resting period, and a testing period on cancerous, precancerous moles and/or precancerous lesions. The physician should first ascertain whether or not the suspect site is precancerous or cancerous using the standardized indicators for skin cancer. Once the physician has identified those cells that have become pre-cancerous or cancerous cells, a single topical administration of the composition of the present invention should be administered to the affected area. Remission is generally obtained within 10 to 12 days and usually about 3 to 6 days. The composition is fast acting and long lasting and has substantial cosmetic benefits for the patient, particularly for the skin on a patient's nose and ears.

If sufficient results are not obtained within 6 to 8 days after initial topical administration, the composition of the present invention may be re-administered after 6 to 8 days. The majority of patients will complete the course of the topical therapy treatment within 3 to 6 days. Bio-markers will show tumor suppression efficacy on an individual patient within the first 48 hours following the initial topical administration.

The topical compositions of the present invention include cesium and/or rubidium ions, preferably in an amount sufficient to provide from about 5,000 ppm to about 100,000 ppm, and more preferably about 10,000 ppm to about 50,000 ppm. The inclusion of rubidium and/or cesium salts is beneficial, as the corresponding cesium ions will eliminate pain. Cesium salts are preferred as the source of cesium ions. The topical composition of the present invention may also include a soothing agent or agents such as menthol or mentholyptus compositions. In addition, the composition of the present invention may also include Vitamin B6 (pyridoxine) and/or Vitamin B12 with a surfactant or surfactants. The non-cesium/rubidium components must be non-bonding with the cesium and/or rubidium ion.

For most topical applications, the optimum pH for the skin without causing irritation is about 7.10. Depending on age and gender and condition of the skin, the pH of the inventive composition should range between 6.70 and 7.10. The composition of the present invention has a preferred pH range of about 6.70 to 6.80 for patients with very thin or fragile skin, and in particular for patients of advanced age. Even a slight pH change in some sensitive patients can irritate their skin. The patient should not expose the affected area or areas to sunlight during the therapy cycle.

It may be necessary to penetrate below the first or second layer of the skin to reach tumors or serious lesions that may metastasize and are life threatening to the patient. In this case, the preferred viscosity range of the topical composition is between about 15 to 25 dynes per cm². For topical applications, the surface tension is adjusted such that the composition of the present invention penetrates through and just a little below the first three dermal layers, thus producing minimal leakage of the cesium ions thereby eliminating damage to the surrounding healthy, viable cells.

The viscosity of the inventive composition is preferably adjusted to sufficiently penetrate through the first and, if necessary, the third layer of skin, to the subdermal tissues and into the cancerous tumors, but not excessively deep so as to penetrate the underlying vasculated tissue. The composition may comprise a small amount of Dimethyl Sulfoxide (DMSO), ranging between 0.20% to about 1.50%, preferably about 0.75% with a maximum of about 5.0%, for adjusting the viscosity to a range that promotes the transport of the salts comprising cesium and/or rubidium ions into or between the cancerous cells.

If necessary, chelation therapy may be administered in conjunction with administration of the inventive composition. Patients who previously received cesium or rubidium therapy, sensitive patients (e.g. advanced age) who have previously been administered multiple cesium and/or rubidium topical therapies, and patients who have received excessive doses (such as oral, injection or other routes of delivery) of the inventive composition over a short period of time may all be candidates for chelation therapy.

In a preferred embodiment of the present invention it is preferred to avoid surfactants that contain any toxic substances such as toxic petroleum based chemicals that may be transported into the healthy cells or may produce other toxic compounds.

### EXAMPLE 1

Topical Protocol

A formula containing cesium chloride in olive oil was topically administered for 8 consecutive days to a 62-year-old male having a large suspect mole on his back approximately 18 mm by 35 mm. The mole was completely removed in about 10 days, and the underlying skin looked healthier and younger than the surrounding skin.

### EXAMPLE 2

Topical Protocol

A formula containing cesium chloride with water was topically administered for 7 consecutive days to a 56-year-old male having a large suspect flat mole on his scalp approximately 10 mm by 12 mm. The mole was completely removed in about 10 days, and the underlying skin looked healthier and younger than the surrounding skin.

### EXAMPLE 3

Topical Protocol

A formula containing rubidium carbonate in sesame oil was topically administered for 7 consecutive days to a 63-year-old male having 6 suspect round moles on his neck approximately 4 to 5 mm in diameter. The moles were completely removed in about 8 days.

Topical Wash/Rinse

The topical composition may be administered as a wash or rinse to suppress tumor growth after surgical procedures, to obtain remission, or to suppress secondary effect of bacterialogical invasion. The surface tension of the inventive composition when used in the form of a wash or rinse is may be adjusted using a surfactant ranging between 8 to 40 dynes per cm², and more preferably a surfactant ranging from 15 to 25 dynes per cm².

Promote Healing Cycle

The methods and compositions of the present invention encompass stimulating the healing process of any wound(s) contaminated with microorganisms. The compositions of the present invention function specifically to maintain the necessary antibacterial environment for wounds to heal faster, without the usual complications associated with superficial infections. In addition, the solutions provide pain suppression and less scarring.

Wound Care

In one preferred embodiment of the present invention the method and composition encompasses preventing and suppressing a broad spectrum of microbial growth (anti-microbial) and reducing inflammation in the targeted zone, thereby promoting faster wound healing, especially in the area of more efficient delivery of the therapeutic agent to the wound care site and/or tumor site, particularly in the topical application to wounds, burns, etc. Cesium salts are preferred. The topical application most preferred is the combination of cesium sulfate and cesium citrate.

This exemplary embodiment of the present invention encompasses administering the inventive composition in the form of a treated bandage or gauze for a wide variety of wound treatments. An exemplary embodiment of the current invention encompasses a method of using the inventive composition as an adjunct to other topical treatments, including, but not limited to, bandages that may be used in combination with the composition or with other compositions or topically applied materials for improvements in wound care management using a combination or mixture of cesium and/or rubidium salts and/or a specific combination of cesium and/or rubidium salts with improved anti-microbial and anti-inflammatory compositions and wound dressings. The cesium and/or rubidium salts preferably comprise a mixture of cesium sulfate and cesium citrate.

The inventive composition may be topically administered with a pH ranging between 6.50 to 7.20, more preferably between about 6.70 to 7.10, and with an ORP ranging between −1 m.v. to −50 m.v., more preferably about −20 to −25 m.v. A suitable dose of the composition is administered to affect the injured sites and cells in order to establish an electro-minus charge. This promotes the electrical flow and simultaneously reduces scarring and pain while suppressing infection.

The composition suppresses infections that occur during surgery and shortens recovery periods and promotes the healing cycle of the normal, healthy, viable cells. In addition, the residual tumor cells will encounter a more hostile environment (more alkaline pHe) and, as a consequence, they are rendered nonviable and are eliminated.

The invention also relates to a method of treating burns and wounds and the use of the subject compounds as antimicrobial agents.

Antimicrobial properties of the composition include, but are not limited to, preventing growth of Escherichia coli, Listeria monocytogenes, Staphylococcus aureus, methicillin-resistant S. aureus (MRSA), Pseudomonas aeruginosa, Lactobacillus, yeast, vancomycin-resistant enterococcus, molds, and spores. The compositions of the invention may be osmotically balanced and of minimal cytotoxicity.

The application of a negative charge promotes the increase of the electrical current density to certain types of wounds and tumors. This increases blood flow to the wound thereby killing a wide variety of pathogens at an increased rate and minimizing the likelihood of prolonged infection, tissue swelling, and pain.

In one exemplary embodiment of the current invention a method and composition contains a formula of cesium and/or rubidium salts and minerals, such as an electrolyte concentration in an isotonic state. The solution's composition typically comprises halide salts of sodium, potassium, calcium, and other cations and most typically chloride, sodium, potassium, and magnesium in the solution. The composition of the invention is atoxic and has antibacterial properties.

Another exemplary embodiment of the present invention includes a cesium and/or rubidium salt containing composition having cesium citrate adjusted to a near neutral pH with a concentration of 1/10 of one percent and a viscosity that is below 33 dynes/per cm², more preferably ranging between 15 to 20 dynes per cm².

The composition is useful in any application in which antimicrobial properties are desirable. Such applications include, without limitation, treatment of wounds, burns, and canker sores; irrigation; cleaning of tissue sites (e.g., pre- and post-operative); for dermatological applications, psoriasis; and numerous applications which are readily apparent to one skilled in the art.

Wound Care Example

A formula containing cesium chloride and olive oil was topically administered for 3 consecutive days to a 42-year-old male having a slow-healing wound on his shin approximately 5 to 6 mm in diameter. The wound healed in about 4 days, and the underlying skin looked pink and younger than the surrounding skin.

Scarring

The composition may be incorporated into bandages as an anti-bacteriant to reduce the scar formation process. The composition promotes the healing of the affected tissue from the inside of the tissue. The undamaged normal healthy viable cells are protected by the osmotic shielding of the cells, and the damaged unshielded or partially unshielded cells disassociate and are metabolized thereby reducing the formation of scar tissue and bacteria infection. The composition is particularly useful in topical applications.

Surgical Procedures

The invention encompasses administering the composition as an adjunct with surgical procedures for reducing the amount of scar tissue formation during surgical procedures. Scar tissue occurs when there is an incorporation of large quantities of damaged cells in the formation of tissues. This often occurs when the immune system is enveloped in the process of healing tissues by the infected cells at the site of tissue damage. The cesium ions in the composition promote the production of less defective cells and thus less scarring, i.e. there are less defective or damaged cells to be enveloped and this enhances the tissues' healing process at the site of damage and potentially lessens the potential future transformation that is necessary for cancer reoccurrence. The cesium ions in the composition also eliminate the electrophysical environment in the scar tissue that promotes metastasis.

The invention encompasses all pharmaceutically active species of cesium and/or rubidium salts and also encompasses hydrated versions, such as solution(s), hydrolyzed products or ionized products of these compounds. These compounds may contain different numbers of attached water molecules. The invention includes any cesium and or rubidium salt or salts or compound(s), whether used alone or in combination with other compounds, that can alleviate, reduce, ameliorate, prevent, or place or maintain in a state of remission the clinical symptoms or diagnostic markers associated with cancers.

In one preferred embodiment the current invention may take the form of a composition and method used as an adjunct with a wide variety of procedures, such as, but not limited to, electrodesiccation and curettage, cryosurgery, simple wide excision, micrographic surgery or laser therapy. The method and composition of the present invention may also be used sequentially with other treatments that are used when the cancers are detected at a later stage of development. These treatments include external radiation therapy, chemotherapy, and to a lesser extent bio-immunotherapy or photodynamic therapy. Sequential administration is preferred, i.e. the method and composition of the present invention is preferably administered sequentially after the other treatments.

Laser Therapy

The present invention encompasses using the method and composition of the present invention as an adjunct with laser therapy. The method and composition of the present invention may be used as an adjunct with laser therapy to reduce the scarring and pain that occurs as a result of wounds and/or surgical procedures etc. and to simultaneously suppresses staff infections and to promote and stimulate the healing process.

Radiation Pellets

The method and composition of the present invention may also be administered to a patient diagnosed with cancer such as, but not limited to, prostate and breast cancers, as an adjunct with implanted micro-radiation-pellets thereby providing a significant improvement over the prior art of radiation therapy alone. The topical composition of the present invention may be simultaneously or sequentially administered.

Biopsy Procedures

The method and composition of the present invention may also be used as part of the protocol for a biopsy procedure prior to, during, or after anti-cancer and/or anti-metastasis therapy. For example, the topical composition of the present invention may be topically administered to a patient preceding the taking of a tissue sample biopsy to prevent the risk of promoting metastasis during the procedure(s).

Cryogenic Therapy

The present invention encompasses using the method and topical composition of the present invention as an adjunct for cryogenic procedures for consolidating topical cancer remission. The topical composition is administered to the affected area 2 to 5 minutes after cryogenic procedures (after the temperature returns to normal) and the affected area is then covered with a bandage.

Chemotherapy Protocol

The method and topical composition of the present invention may be used for the treatment and prevention of metastases and cancer recurrences after operations, radiotherapy and chemotherapy, in an effort to consolidate remission.

A chelation protocol may also be used as part of the treatment. As an example, Vitamin E doses may be administered as a catalyst to a 70 kg male for 24 hours (with a maximum dose of about 20 i.u.) in addition to Omega 3 oils, and/or other fish oils that are suitable as bonding agents or for promoting bonding neutrality. The maximum dose is 10× the daily recommended dose ranging between 3× to 10× the daily dose in sufficient quantities to promote and eliminate the toxins from the patient's system that are encountered from the chemistry poisons and or radiation poisons produced during the chemotherapy and radiation therapies.

The physician may administer the composition as an adjunct for chemotherapy. For example, the topical composition of the present invention may be administered sequentially between about 1 to 4 hours after the initial administration of the chemotherapeutic agent or agents (depending on the chemistry of chemotherapeutic agents and the variety of cancer) in order to provide a sufficient time interval for the chemotherapeutic agents to initiate and poison the cancer cells. The inventive topical composition and the chelating composition block some of the harmful effects of the chemotherapy from the normal healthy viable cells thus promoting therapeutic gain and reducing a variety of side effects.

The present invention also encompasses administering the topical composition to a cancer patient to shorten the therapeutic regime of a chemotherapy treatment schedule and to potentially lower the patient's overall doses thereby reducing the systemic toxicity from the chemotherapeutic agent(s).

The topical composition of the present invention has the potential to elevate (amplify) the anti-cancer activity of the chemotherapy by up to about 30%.

Direct Tumor Injection

In another exemplary embodiment of the present invention, the method and composition encompasses administration by direct injection into a tumor mass with a syringe or other suitable delivery method encompassing directly injecting into the tumor mass to improve or enhance site directing or targeting. The composition is pH balanced and acceptable for injection, encompassing a protocol that is followed by a resting period, then clinically testing for verification of tumor shrinkage after about 24 to 48 hours time. The compositions includes cesium and/or rubidium salt in a solution having an adjusted surface tension ranging from about 8 to 15 dynes per cm², preferably ranging between 8 to 15 dynes per cm², more preferably about 10 dynes per cm² and producing an O.R.P. from −1 m.v. to −100 m.v., and preferably between −10 m.v. to −70 m.v.

The direct injection is administered sequentially or simultaneously with vitamin B12. It may also administered with nutrient support composition including, but not limited, one or more of Vitamin D2, Vitamin D3, Vitamin B6 with Omega 3 oils, Vitamin A, and other nutrient support and sequentially administering chelators.

Generally, the duration from direct injection into the tumor mass to promote complete remission is obtained within 10 to 15 days from the initial tumor injection. Surfactants may also be formulated with the inventive composition that are specifically tailored to the type or variety of cancer.

Stomatitis and Mucositis

The invention also encompasses a method and composition for the treatment of stomatitis and/or mucositis with an oral rinse or wash. The composition penetrates the cancer or precancerous regions including the gums to obtain their anti-tumor, anti-inflammation, anti-pain, and anti-microbial activity.

The present invention provides an ionic, pH manipulating composition and protocols for inhibiting acidotic activity (reduced pH) for prophylaxis of a wide variety of oral diseases including precancerous lesions and a variety of oral cancers. The method and composition are effective to minimize levels of pain and symptoms that occur in a reduced pH environment, particularly in the region of the mouth and throat.

Radiotherapy and Chemotherapy

The method and composition of the present invention encompasses a treatment of cancer using cesium and or rubidium as an adjunct with other anti-cancer agents. Use of the method and composition of the present invention lessens the risk of causing cancer in a patient's normal healthy cells when radiation therapy is used to treat cancer cells, and reduces or eliminates the suppression of the immune response that often occurs in conventional cancer therapies.

The present invention encompasses, but is not limited to, the sequential or simultaneous administration of a cytotoxic chemical compound or compounds such as, e.g., a nitrosourea, cyclophosphamide, adriamycin, 5-fluorouracil, paclitaxel and its derivatives, cisplatin or other cancer treating agents in combination with the topical composition of the present invention. The cytotoxic composition may be administered by any suitable delivery route.

Administering the composition of the present invention as an adjunct with a variety of chemotherapeutic agents is useful in the treatment of neoplastic disease. It also enhances the tissues' healing process at the site of the damage and lessens the potential future transformation that is necessary for cancer reoccurrence. It also prevents a metastasis environment in scar tissues, and prevents cancer recurrences after operations, radiotherapy and chemotherapy.

The present invention provides a method of chemosensitization which includes administering a chemotherapeutic agent and a composition to a patient. “Chemosensitization”, as used herein, means that the composition increases or enhances the cytotoxicity of a chemotherapeutic agent or agents compared to a level of cytotoxicity seen by that agent in the absence of the inventive composition. That is, the composition “sensitizes” a cancer cell to the effects of the chemotherapeutic agent, allowing the agent or agents to be more effective. The composition is also known to have anti-cancer activity on its own.

The compositions of the present invention may be used as stand alone therapies or in combination as an adjunct with other known therapeutic agents (including chemotherapeutics, radio-protectants and radio-therapeutics) or techniques to either improve the quality of life of the patient, or to treat cancer. For example, the cesium salt compounds can be used before, during or after the administration of one or more known antitumor agents including but not limited to a wide variety of mustard compounds. (See, for example, the Physician Desk References 1997). In addition, the cesium and or rubidium salt compounds can be used after radiation treatments. The composition shortens the therapeutic window for the radiation therapy.

The novel composition is useful as chemo-sensitizers or radio-sensitizers in patients having cancer. Thus, the compounds or composition have utility in patients having cancer which are sensitive to radiation or chemotherapy. The composition is typically administered to patients after they are subjected to irradiation or chemotherapy administration.

Cesium ions are alkaline chemotherapy agents that disrupt the DNA of cancer cells. One exemplary embodiment of the present invention encompasses administering the inventive composition to block the tumor cell's ability to repair themselves after damage by chemotherapy and/or radiation, thus promoting the enhancement of tumor necrosis from radiation or chemotherapy, particularly tumor response to weak base drugs.

The composition of the present invention enhances the uptake of the composition drugs into cancer cells. This chemistry usually includes weak acids or weak bases, is limited under conditions due to the acidic extracellular/interstitial environment and neutral to basic intracellular conditions and the tumor's micro-circulatory factors. The pH gradient of the cancer cells' microenvironment is elevated, thus enhancing the uptake of the chemotherapeutic agents particularly the weak acids or the weak bases in the target tumor cells.

An exemplary embodiment of the present invention includes a method of treating cancer in a patient including the steps of administering to the subject a chemotherapeutic agent and then administering the composition containing cesium and or rubidium salts having radiosensitization properties. The composition is administered after ionizing radiation to the patient in proximity to the cancer. Cesium and rubidium have radiation sensitization properties, such as, but not limited to, enhanced removal of toxins from ionizing radiation. Ionizing radiation includes, but is not limited to, x-rays, internal and external gamma emitting radioisotopes, and ionizing particles.

Adjunct for Topical Chemotherapy

In another aspect of the invention, cesium and or rubidium may be used as an adjunct for topical chemosensitizers. For example, the composition may be topically administered sequentially to a patient suspected or diagnosed with cancer. The use of cesium and or rubidium is used to enhance the cytotoxicity of topically administered chemotherapeutic agents or as a stand alone chemotherapeutic agent. Chemosensitization using cesium and or rubidium refers to an enhancement of cytotoxicity on the part of a chemotherapeutic agent more preferably comprising a combination of cesium salts, such as sulfate and citrate.

The method comprises the steps of i) assaying cytotoxicity of a candidate chemotherapeutic agent in the presence and in the absence of cesium and or rubidium, and ii) selecting a candidate chemotherapeutic agent as a chemotherapeutic agent for which cesium and or rubidium salt or salts is a chemosensitizer when the cytotoxicity of the candidate agent is greater in the presence of cesium and or rubidium than in the absence of cesium and/or rubidium. This may be accomplished by the administration of the composition then waiting 12 to 48 hours, more preferably 24 to 48 hours, and then testing (evaluating) and verifying the tumor control (shrinkage) by methods know in the physicians' art. The same procedure may be employed for other routes for radiosensitizing agents or chemosensitizing agents, or as a stand-alone chemotherapeutic, etc.

In one exemplary embodiment of the present invention, the chemotherapeutic composition can be incorporated into topical creams, lotions, solutions, ointments, gels, and other dermatologic formulations for topical treatment of skin cancer.

In another exemplary embodiment of the present invention, the method of the present invention encompasses administrating the topical composition to a patient who is diagnosed or is suspected of having cancer, such as, but not limited to, squamous cell carcinoma, basal cell carcinoma, and cutaneous lymphoma, and for premalignant lesions such as actinic keratoses, and lesions of the skin such as psoriasis, seborrheic keratoses, and discoid lupus erythematosus. The physician should topically apply the dermatologic formulation only to the affected areas. The required duration of occlusion is variable, depending on the stage of advancement, such as, for example, the thickness of the cancerous lesions and the presence of thick scales, etc.

Cesium used as chemosensitizers or radiosensitizing agents may be administered sequentially after administration of the chemotherapeutic agent or agents or after about 2 to 4 hours following administration of the radiotherapy. The cesium containing composition is preferably used after 2 to 5 hours and more preferably after 2 to 3 hours following administration of the radiotherapy.

A time frame or treatment schedule for in vivo administration of the composition is about 2 to 5 hours after administration of the chemotherapeutic agent. As an example, when administering a therapeutic dose to a 70 kg male, the physician should consider the patients' activities and the foods and food products the patient is ingesting including diet, the variety of cancer, stage of advancement and the total volume and mass of the cancer as well as how it is distributed in the body of the patient.

The composition to be used in the method of the invention will be administered in a pharmaceutically effective amount. By “pharmaceutically effective” it is meant that the dose which will provide an enhanced toxicity to radiotherapy, and a variety of chemotherapeutic agents. The specific dose will vary depending on the particular composition chosen, the dosing regimen to be followed, when the particular chemotherapeutic agent is administered, and the route of administration.

The present example provides methods for selecting chemotherapeutic agents for which the composition is a chemosensitizer. The candidate chemotherapeutic agents are screened for enhanced activity in the presence of the composition (cesium and/or rubidium salts) using an in vitro cytotoxicity assay such as the MTT cytotoxicity test. The composition is considered a chemotherapeutic agent for which the inventive composition is a chemosensitizer. The drug resistance may be circumvented in a dose-dependent manner by simultaneous or sequential administration of the present composition such as cesium and/or rubidium. Additionally or alternatively, a MDR1-transgenic mouse model may also be used to test for those chemotherapeutic agents for which compositions of the present invention is a chemosensitizer in repressing drug resistance.

The relief offered by the compositions is typically long-lasting, such that the reduction or elimination in pain occurs quickly and for a substantial period of time. The inventive composition containing cesium and/or rubidium salts may be administered as a topical chemotherapy to a patient diagnosed with such cancers.

Chemotherapy Adjunct

An exemplary embodiment of the present invention encompasses administering the composition as an adjunct with chemotherapy. The cesium amplifies the chemotherapy that a patient has received or is going to receive. The maximum dose regimes for the therapy should not exceed 60 days consisting of three 5-day regiments followed with a resting and testing period of 15 days per each 5 days of consecutive administration.

The composition may be administered as a stand-alone therapy or as an adjunct radiotherapy and chemotherapy preferably administered sequentially with a composition containing of Vitamin D2, VitaminD3, and B6 (pyrodoxine).

In another exemplary embodiment of the invention, the method and composition encompasses administering a variety of cesium salt with doses ranging from between 1,200 mg to 2,500 mg per 24 hours for a 70 kg male sequentially with a composition containing vitamin D2 and D3 with dosage ranging between (0.33 to 4.00 mg/kg), co-administered with Vitamin B6 (pyridoxine) with dosages ranging between 1 mg to 40 mg per 24 hours for a 70 kg male. The topical composition may contain a suitable surfactant to adjust the viscosity within the therapeutic range from about 40 dynes/cm² to 10 dynes per cm² more preferably ranging between 10 to 20 dynes/cm² to effectively penetrate the nucleus of the cancerous cells.

The invention also encompasses the use of combination therapy to treat cancers, especially cancers which are refractory to other forms of treatment. In accordance with the present invention, the composition can be used alone or in combination with other known therapeutic agents (including chemotherapeutics, radioprotectants and radiotherapeutics) or techniques such as surgery procedures, curettage, leatrile therapy, radiation therapy, immuno-therapy, implanted radiation pellets and laser therapy or enzyme therapy to either improve the quality of life of the patient, or to treat cancer. The compositions of the current invention can be used before, during or after the administration of one or more known chemotherapeutic agents. In addition, the composition can be used before, during or after radiation treatment sequentially after radiation treatment is preferred.

Pain Management

In one embodiment of the present invention encompasses a method for administering the composition for suppression and elimination of tumor generated pain.

When administered, the composition provides for antimicrobial activity. This activity reduces or eliminates the acidotic environment that promotes pain and lesions and will quickly reduce or eliminate tumor-generated pain for extended periods of time.

Wound Care Kit

In one embodiment of the present invention, the invention also provides kits for carrying out the therapeutic regimens of the invention. Such kits comprise in one or more containers therapeutically effective amounts of the composition in pharmaceutically acceptable form. The composition in a vial of a kit of the invention may be in the form of a solution, e.g., in combination with sterile saline, or buffered solution, dextrose solution or other pharmaceutically acceptable sterile fluid. Alternatively, the composition may be lyophilized or desiccated; in this instance, the kit optionally further comprises in a container a pharmaceutically acceptable composition, preferably sterile, and/or a packaged alcohol pad. Instructions are optionally included for topical administration of the composition by a physician.

Reverse Catheter

Administration of the composition of the present invention by reverse catheter or other suitable method is also contemplated by the present invention. The method and composition of the present invention may be used to treat bladder and renal cancers having measurable tumor-selective alkalinization. Use of the composition may enhance tumor response to weak base drugs with significant alkalinization of urine in humans for enhancement of a variety of anti-cancer therapies for certain bladder and renal tumors that are treated using weak base drugs such as but not limited to, anthracycline and vinca that enhanced therapeutic gain in a weak base environment.

Cancer cells tend to have thinner cell membranes than normal, healthy cells. This factor has been the foundation of numerous types of chemo- and radiation therapies, which operate on the principle that cancer cells tend to be more susceptible to death due to cell stress and damage than normal cells. Medical research has not currently found a way to exploit the very different metabolic distinctions between cancer cells and healthy cells: the fact that cancerous tumors tend to undergo anaerobic glycolysis and generate more acidity than normal, healthy cells. This invention, rather than inflicting serious damage on all of the cells in the body in an effort to selectively target and kill cancer cells, provides a more benign approach to treating cancer particularly glycolysis dependent tumors or tumor that have tumor microenvironments regardless of tumor type.

One example in which almost all cancer cells differ from normal healthy cells is that cancer cells derive a major proportion of their energy from glycolysis. Normal healthy cells utilize an oxidative metabolism in which only a small proportion of energy is derived from glycolysis. Cancerous neoplasms require an alteration of this energy production with a transition from non-invasive premalignant to invasive malignant morphology, ranging from large benign tumors to necrotic cancers, including the acquisition of angiogenesis, increased glucose utilization (with increased lactic acid production) with localized tissue acidity and typical tumor morphologies.

The increased lactic acid production of tumors causes the micro environment outside the tumor to become more acidic, leading to a reduced acidic pH. This acidic pH kills the normal tissue cells, which surrounds the tumor (note the normal viable cells require a mild alkaline pHe of about 7.31 or slightly higher to remain healthy and viable.) As a consequence, the tumor is surrounded by necroticised normal cells. If insufficient alkalinizing agents are available to the healthy tissue cells surrounding the tumor, this promotes the extension (“invasion”) of the tumor into normal tissues. Increasing tissue permeability and the formation of new blood vessels (angiogenesis).

Additionally, the values/ranges of pHe measured (the pH of the immediate environment of the tumor), vascularization, angiogenesis and surrounding tissue permeability correlate with invasiveness and metastasis and this (acidic) sub optimal-physiological pHe makes tumor cell lines more metastatic.

Cancerous viability is dependent on an acidic micro-environment. This is due in part to their aberrant energy metabolism which produces lactic acid and carbonic acid and in part to incomplete vascularization, which causes insufficient oxygen supply (hypoxia).

Tumors tend to be both hypoxic and acidic. Chronically hypoxic tissues are going to be (i.e. are always) acidic, whereas transiently hypoxic tissues may be acidic. This is due to microcirculatory factors, the more central part of the tumor is hypoxic, the exterior is transiently hypoxic.

The common denominator for nearly all solid tumors (neoplasms) is an acidic pH at the tumor's edge. This tumor pHe referred to as the tumor-micro environment that has a pH that ranges from as low as 5.50 to up to about 7.20 with an optimum cancer viability pH ranging between about 6.60 to 6.85.

The Cesium and/or rubidium ions selectively alkalinize tumors by site affinity increasing the buffering capacity of tumor interstitial fluid, with only minor pH elevation effects on the pHe of normal, healthy, viable cells and tissues. Cesium ions in the present invention are separate and distinct from man-made isotopes of cesium and rubidium.

The facts and theories discussed in this disclosure are intended to teach the physician how to use the invention. While this invention has been described in connection with preferred embodiments, it is obvious that various modifications, changes or substitutions therein may be made by those skilled in the art to which it pertains, without departing from the spirit and scope of the invention. Accordingly, the scope of the present invention is to be limited only by the appended claims and their legal equivalents. The above disclosure is sufficient to enable one of ordinary skill in the art to practice the invention, and provides the best mode of practicing the invention presently contemplated by the inventor. While there is provided herein a full and complete disclosure of the preferred embodiments of this invention, it is not desired to limit the invention to the exact construction, dimensional relationships, and operation shown and described. Various modifications, alternative constructions, changes and equivalents will readily occur to those skilled in the art and may be employed, as suitable, without departing from the true spirit and scope of the invention. Such changes might involve alternative materials, components, compositions, compounds, method steps, order, sequence, structural arrangements, functions, or the like.

1. An anti-cancer composition for topical administration comprising:
at least one of a cesium ion source and a rubidium ion source; and
a carrier suitable for topical application.

2. The composition of claim 1 wherein the cesium ion source comprises a combination of cesium sulfate and cesium citrate.

3. The composition of claim 1 wherein the composition has a viscosity of at or below 36 dynes per cm².

4. The composition of claim 1 wherein the composition comprises a pH within a range of about 6.7 to 7.2.

5. The composition of claim 1 wherein the carrier comprises a liquid, a gel, a cream, an ointment, a lotion, a paste, an emulsifier, a solvent, a liquid diluent, and a powder.

6. The composition of claim 1 further comprising a surfactant.

7. The composition of claim 6 wherein the surfactant comprises at least one of a polysorbate, a sorbitan stearate, a sorbitan mono-oleate, a sarcosinate, a cetyl pyridinium chloride, a cetyl trimethyl ammonium bromide, a di-isobutyl phenoxy ethoxy ethyldiniethyl benzyl ammonium chloride, a coconut alkyl trimethyl ammonium nitrate, an alkyl sulfate, a fatty alcohol, a fatty amide, a polyhydric alcohol, a polyethylene glycol, a sodium lauryl sulfate, a sorbitan laurate, a sorbita palitate, a polyoxyethylene (20) sorbitan monolaurate, a polyoxyethylene (20) sorbitan monopalmitate, a polyoxyethylene (20) sorbitan monostearate, a benzalkonium chloride, a glycerin, and a DMSO.

8. The composition of claim 6 wherein the carrier and the surfactant comprise glycerin.

9. The composition of claim 6 wherein the surfactant comprises DMSO within a range of about 0.20% to 5%.

10. The composition of claim 1 wherein the composition comprises a viscosity of within a range of about 15 to 25 dynes per cm² and a pH within a range of about 6.7 to 7.2.

11. The composition of claim 1 wherein the composition comprises a viscosity of within a range of about 8 to 20 dynes per cm² and a pH within a range of about 6.7 to 7.2.

12. The composition of claim 1 wherein the cesium ion source comprises a cesium salt selected from at least one of a Carbonate, a Chloride, a Citrate, a Malic, a Malate, a Nitrate, a Phosphate, a Sulfite, a Sulfate.

13. The composition of claim 12 wherein the cesium salt comprises a combination of cesium sulfate and cesium citrate in a ratio having a range of about 2-3 cesium sulfate to about 1 cesium citrate.

14. The composition of claim 12 wherein the cesium salt comprises cesium citrate with a concentration of about 0.001% and the composition comprises a viscosity within a range of about 10 to 25 dynes per cm².

15. The composition of claim 1 further comprising a soothing agent which includes a eucalyptus, a menthol, or a mentholyptus composition.

16. The composition of claim 1 wherein said at least one of a cesium ion source and a rubidium ion source comprises a range of about 5,000 ppm to 100,000 ppm.

17. The composition of claim 1 further comprising an electrolyte comprising at least one of a sodium, a potassium, a calcium, a chlorate, a magnesium, a bicarbonate, a phosphate, and a sulfate.

18. The composition of claim 1 further comprising at least one of an antibacterial agent, an antifungal agent, and an anti mold agent.

19. The composition of claim 1 wherein the composition comprises an ORP within a range of about −1 m.v. to about −50 m.v.

20. The composition of claim 1 wherein the cesium ion source comprises cesium chloride.

21. The composition of claim 1 wherein said composition is used as an adjunct with at least one of a surgical procedure, a laser therapy, a micro-radiation pellet implant, a cryogenic procedure, a chemotherapy, a radiation therapy, and a electrodessication/curettage, a laetrile therapy, an immuno-therapy, and an enzyme therapy.

22. A method for preventing or treating cancer comprising the step of topically administering the composition of claim 1.

23. A method for treating at least one of a wound, a sore, a pain, an infection, an inflammation, a scar, and a dermatological condition comprising the step of administering the composition of claim 1.

24. A composition for enhancing the toxicity of a topically administered chemotherapeutic agent comprising:
at least one of a cesium ion source and a rubidium ion source; and
a carrier suitable for topical application.

25. The composition of claim 24 wherein the composition has a viscosity of at or below 36 dynes per cm².

26. The composition of claim 24 wherein the composition comprises a pH within a range of about 6.7 to 7.2.

27. The composition of claim 24 wherein the carrier comprises a liquid, a gel, a cream, an ointment, a lotion, a paste, an emulsifier, a solvent, a liquid diluent, and a powder.

28. The composition of claim 24 further comprising a surfactant.

29. The composition of claim 24 wherein the cesium ion source comprises a cesium salt selected from at least one of a Carbonate, a Chloride, a Citrate, a Malic, a Malate, a Nitrate, a Phosphate, a Sulfite, a Sulfate.

30. The composition of claim 24 further comprising a soothing agent which includes a eucalyptus, a menthol, or a mentholyptus composition.

31. The composition of claim 24 wherein said at least one of a cesium ion source and a rubidium ion source comprises a range of about 5,000 ppm to 100,000 ppm.

32. The composition of claim 24 further comprising an electrolyte comprising at least one of a sodium, a potassium, a calcium, a chlorate, a magnesium, a bicarbonate, a phosphate, and a sulfate.

33. The composition of claim 24 further comprising at least one of an antibacterial agent, an antifungal agent, and an anti mold agent.

34. The composition of claim 24 wherein the composition comprises an ORP within a range of about −1 m.v. to about −50 m.v.

35. The composition of claim 24 wherein the cesium ion source comprises a combination of cesium sulfate and cesium citrate.

36. The composition of claim 24 wherein the cesium ion source comprises cesium chloride.

37. The composition of claim 28 wherein the carrier and the surfactant comprise glycerin.
